(19) United States
(12) Patent Application Publication
(10) Pub. No.: US 2023/0240873 A1
(43) Pub. Date: Aug. 3, 2023

(54) RETRACTABLE OUTER SHEATH FOR DRUG COATED BALLOON

(71) Applicant: KONINKLIJKE PHILIPS N.V., EINDHOVEN (NL)

(72) Inventors: Mitchell ERICKSON, CRYSTAL, MN (US); Arjen VAN DER HORST, TILBURG (NL); Roland Alexander VAN DE MOLENGRAAF, GELDEOP (NL)

(21) Appl. No.: 17/785,230

(22) PCT Filed: Dec. 21, 2020
(86) PCT No.: PCT/EP2020/087357

§ 371 Date: Jun. 14, 2022

Related U.S. Application Data
(60) Provisional application No. 62/951,311, filed on Dec. 20, 2019.

(51) Int. Cl. A61F 2/958; A61M 25/00

(52) U.S. Cl. CPC A61F 2/958; A61M 25/0067; A61M 2025/0058

(57) ABSTRACT

The present disclosure relates generally to an outer sheath and the use of the outer sheath with a drug coated balloon for the treatment of vascular conditions. In particular, the present disclosure provides an outer sheath that is capable of covering the expandable member during delivery and removal of the drug coated balloon.

### CROSS REFERENCE TO RELATED APPLICATIONS

The present application claims priority under 35 U.S.C. § 119(e) and under 37 C.F.R. § 1.78(a) to commonly owned U.S. Provisional Application No. 62/951,311 filed on Dec. 20, 2019. The entire disclosure of U.S. Provisional Application No. 62/951,311 is specifically incorporated herein by reference.

### FIELD

The present disclosure relates generally to an outer sheath and the use of the outer sheath with a drug coated balloon for the treatment of vascular conditions. In particular, the present disclosure provides an outer sheath that is capable of covering the expandable member during delivery and removal of the drug coated balloon.

### BACKGROUND

Angioplasty, also known as balloon angioplasty and percutaneous transluminal angioplasty, is a minimally invasive, endovascular procedure to widen narrowed or obstructed arteries or veins, typically to treat arterial atherosclerosis. A balloon catheter having a catheter shaft and an expandable member (i.e., balloon) is used to perform an angioplasty procedure. A physician uses medical imaging to guide the balloon portion of the catheter to the narrowing or obstruction in the vasculature. The balloon is inflated to open the vasculature and improve blood flow. Angioplasty may be done with or without a metal mesh tube called a stent, which if used is left inside the vasculature to assist in keeping the narrowing or obstruction open.

Angioplasty procedures can be used to treat peripheral vascular disease and coronary arterial disease. Peripheral vascular disease refers to diseased blood vessels in a subject's vascular system away from the subject's heart and brain. Although peripheral vascular disease can occur within a subject's arteries (arterial system) or veins (venous system), peripheral vascular disease typically occurs in a subject's arterial system and often in the legs.

Drug-Coated Balloon (DCB) angioplasty is similar to a conventional angioplasty procedure, but there is the addition of an anti-proliferative medication coating on the balloon, as well as an excipient to aid in drug transfer, which may help prevent restenosis. Restenosis is the re-narrowing of the vasculature at a site that was previously treated. Using a drug-coated balloon has the potential to prohibit cell division, limiting the amount of restenosis, or blockage re-growth after treatment.

The balloons of known DCB catheters are delivered to the vascular site in an uncovered state. And if covered during delivery to the vascular site, the balloon is uncovered during removal. When uncovered during either delivery or removal, some of the drug coating on the balloon may be lost and shed downstream within the vasculature. What is needed, therefore, is a device that can cover the balloon of a DCB catheter during delivery to and removal from the vascular site so as to prevent or reduce drug loss from the balloon.

### SUMMARY

The present disclosure discloses an outer sheath that can cover the balloon of a DCB catheter both during delivery to and removal from the vascular site so as to prevent or reduce drug loss from the balloon.

In accordance with a representative embodiment, a method for treating a restriction within a subject's vascular system of the present disclosure comprises: locating a restriction in the vascular system of the subject; positioning a sheath and a balloon catheter within the vascular system of the subject approximate the restriction, wherein the balloon catheter comprises a shaft and an expandable member, wherein the sheath comprises a distal portion and a distal end and an orifice at the distal end and a lumen extending proximally from the orifice, wherein the orifice has a first diameter; positioning the expandable member distally of the distal end of the sheath and adjacent the restriction within the vascular system; expanding the expandable member adjacent the restriction; deflating the expandable member; radially enlarging the orifice at the distal end of the sheath to a second diameter, wherein the second diameter is greater than the first diameter; inserting the expandable member into the distal end of the sheath while the orifice has the second diameter; and radially reducing the orifice at the distal end of the sheath.

In accordance with another representative embodiment, the expandable member comprises a length, wherein the distal portion of the sheath comprises a predetermined length, whereupon inserting the expandable member into the distal end of the sheath, the length of expandable member is disposed within the lumen of the sheath.

In accordance with another representative embodiment, the distal portion of the sheath comprises a predetermined length, whereupon inserting the expandable member into the distal end of the sheath, the length of expandable member is disposed within the predetermined length of the distal portion of the sheath.

In accordance with another representative embodiment, the predetermined length of the sheath is greater than the length of expandable member.

In accordance with another representative embodiment, the radially reducing the orifice at the distal end of the sheath comprises radially reducing the orifice at the distal end of the sheath to a third diameter.

In accordance with another representative embodiment, the third diameter is less than the second diameter.

In accordance with another representative embodiment, the third diameter is substantially the same as the first diameter.

In accordance with another representative embodiment, the method further comprises the step of simultaneously removing the sheath and balloon catheter from the vascular system of the subject.

In accordance with another representative embodiment, a catheter system comprises: a balloon catheter comprising an expandable member; a sheath comprising a distal portion having a distal end, wherein the distal end comprise an orifice having a diameter, wherein the distal portion comprises a lumen extending proximally from the orifice, and a means for increasing and decreasing the diameter of the orifice.

In accordance with another representative embodiment, the catheter system further comprises means for increasing and decreasing the diameter of the orifice comprises a plurality of wires radially disposed within the distal portion of the outer sheath and evenly spaced around a circumference of the distal portion of the outer sheath and extending proximally.

In accordance with another representative embodiment, the outer sheath comprises a handle, wherein the wires are coupled to the handle, and whereupon activating the handle, tension is applied to the wires, and the diameter of the orifice increases.

In accordance with another representative embodiment, when the handle is deactivated, tension is reduced to the wires, and the diameter of the orifice decreases.

In accordance with another representative embodiment, the plurality of wires each comprises a shape-memory metal configured to cause the increasing and decreasing of the diameter of the orifice.

In accordance with a representative embodiment, an apparatus comprises a sheath having a distal portion comprising a distal end. The distal end comprises a first orifice having a first diameter. The sheath comprises an electroactive polymer (EAP), which when actuated, increases the first diameter of the first orifice.

In accordance with another representative embodiment, the sheath has a proximal portion comprising a proximal end, and the proximal end comprises a second orifice having a second diameter. When actuated, the EAP causes no increase or decrease in the second diameter.

In accordance with another representative embodiment, the sheath when actuated has a flared profiled between the first orifice and the second orifice.

In accordance with another representative embodiment the EAP is an ionic EAP.

In accordance with another representative embodiment, the EAP is a field-driven EAP.

The preceding is a summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A is a side view of an exemplary kit or system comprising an outer sheath and a balloon catheter in accordance with a representative embodiment, and illustrates the balloon is in a collapsed state within the outer sheath.

FIG. 1B is a side view of an exemplary system in accordance with a representative embodiment and disposed within a subject's vasculature, illustrating a collapsed balloon extending beyond the distal end of the outer sheath.

FIG. 1C is a side view of an exemplary system in accordance with a representative embodiment, and within a subject's vasculature, with the balloon in an expanded state adjacent the vascular walls beyond the distal end of the outer sheath.

FIG. 1D is a cross-sectional view of an exemplary system within a subject's vasculature, wherein a collapsed balloon extends beyond the distal end of the outer sheath.

FIG. 1E is a cross-sectional side view of an exemplary system in accordance with a representative embodiment, and within a subject's vasculature, with a proximal portion of the collapsed balloon is axially located and extending into the radially expanded distal end of the outer sheath.

FIG. 1F is a cross-sectional view of an exemplary system in accordance with a representative embodiment, and within a subject's vasculature, with the entirety of the collapsed balloon is located within the distal end of the outer sheath.

FIG. 2 is a perspective view of a distal portion of an exemplary outer sheath in accordance with a representative embodiment.

FIG. 3 is a distal end view of the distal end of an exemplary outer sheath in accordance with a representative embodiment.

FIGS. 4A-4D depict sections of a portion of an apparatus comprising an ionic electroactive polymer (EAP) contemplated for use in a sheath in accordance with a representative embodiment.

FIG. 4E is cross-sectional views of an apparatus showing prior to and after actuation of an EAP sheath in accordance with a representative embodiment

FIGS. 5A-5B are perspective views of a sheath comprising EAP prior to and after expansion of a distal portion of the sheath in accordance with a representative embodiment.

FIG. 6 is a cross-sectional view showing a distal portion of a sheath after expansion of an EAP in accordance with a representative embodiment.

FIG. 7 is a representative flow diagram of a method of performing an angioplasty procedure on a subject using the kit or system in accordance with a representative embodiment.

### DETAILED DESCRIPTION

The present teachings relates generally to medical devices for the treatment of vascular conditions. In particular, the present disclosure provides apparatuses, methods and materials for providing an outer sheath that is capable of adapted to cover the expandable member during delivery and removal of the drug coated balloon. However, and importantly, the while various embodiments are described in connection with the insertion, expansion, retraction and withdrawal of balloon catheters, the present teachings are not limited to these applications. Rather, an apparatus comprising a sheath in which an opening or expansion of an orifice is effected is contemplated by the present teachings. For example, an apparatus according to the present teachings may be used in connection with stent retrievers for blood clot removal where opening and closing of the tip (distal portion/distal end) is beneficial for pulling the clot back into the sheath. Additionally, an apparatus according to the present teachings may be used in connection with pulling back embolic protection devices. Furthermore, an apparatus according to the present teachings may be used in connection with opening a tip of laser catheter to allow the laser to open a larger lumen.

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

For purposes of explanation and not limitation, various representative embodiments disclosing specific details are set forth in order to provide a thorough understanding of the present teachings. Other embodiments may be utilized and derived from the disclosure, such that structural and logical substitutions and changes may be made without departing from the scope of the present drawings. Additionally, as noted above, the drawings are merely representational and may not be drawn to scale. Certain proportions within the illustrations may be exaggerated, while other proportions may be minimized. Accordingly, the disclosure and the figures are to be regarded as illustrative rather than restrictive.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (for example, X₁ and X₂) as well as a combination of elements selected from two or more classes (for example, Y₁ and Zₒ).

The terms “about” or “approximately” when used in conjunction with a numeric value shall mean plus and/or minus ten percent (10%) of that numeric value, unless otherwise specifically mentioned herein.

The term “catheter” as used herein generally refers to a tube that can be inserted into a body cavity, duct, lumen, or vessel, such as the vasculature system. In most uses, a catheter is a relatively thin, flexible tube (“soft” catheter), though in some uses, it may be a larger, solid, less flexible—but possibly still flexible—catheter (“hard” catheter). In some uses a catheter may contain a lumen along part or all of its length to allow the introduction of other catheters or guidewires. An example of a catheter is a sheath.

The term “balloon catheter” as used herein generally refers to the various types of catheters which carry a balloon for containing fluids. Balloon catheters may also be of a wide variety of inner structure, such as different lumen design, of which there are at least three basic types: triple lumen, dual lumen and co-axial lumen. All varieties of internal structure and design variation are meant to be included by use of the term “balloon catheter” herein. In some uses, balloon catheters can be used to perform angioplasty.

The term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “sheath” as used herein generally refers to a tube that can be inserted into a body cavity duct, lumen, or vessel, such as the vasculature system that allows for the introduction of other devices, such as catheters, and the introduction of fluid along its length. The sheath can have a closed end or an open end. Because the sheath is a tube that can be inserted into a body cavity, duct, lumen, or vessel, such as the vasculature system, the sheath may also be considered a catheter.

The term “therapeutic agent” as used herein generally refers to any known or hereafter discovered pharmacologically active agent that provides therapy to a subject through the alleviation of one or more of the subject's physiological symptoms. A therapeutic agent may be a compound that occurs in nature, a chemically modified naturally occurring compound, or a compound that is chemically synthesized. The agent will typically be chosen from the generally recognized classes of pharmacologically active agents, including, but not necessarily limited to, the following: analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals;

antihelminthics; antihistamines; antihyperlipidemic agents; antihypertensive agents; anti-infective agents such as antibiotics and antiviral agents; antiinflammatory agents; antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, CNS agents; beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; restenosis inhibitors; muscle relaxants; narcotic antagonists; nicotine; nutritional agents, such as vitamins, essential amino acids and fatty acids; ophthalmic drugs such as antiglaucoma agents; parasympatholytics; psychostimulants; sedatives; steroids; sympathomimetics; tranquilizers; and vasodilators including general coronary, peripheral and cerebral.

The terms “vasculature” and “vascular” as used herein refer to any part of the circulatory system of a subject, including peripheral and non-peripheral arteries and veins. Vasculature can be comprised of materials such as nucleic acids, amino acids, carbohydrates, polysaccharides, lipids fibrous tissue, calcium deposits, remnants of dead cells, cellular debris and the like.

The term “vascular occlusion” or “occlusion” refers to buildup of fats, lipids, fibrin, fibro-calcific plaque, thrombus and other atherosclerotic tissue within the lumen or within the intima of an artery that either narrows or completely obstructs the inner lumen the artery thereby restricting or blocking normal blood flow through the artery segment. The occlusion may partially or totally occlude the vasculature. Accordingly, the term “vascular occlusion” or “occlusion” shall include both a total occlusion and a partial occlusion. Alternatively, a vascular occlusion or occlusion may also be referred to as a vascular obstruction (or obstruction) or a vascular restriction (or restriction). A vascular obstruction may, therefore, be referred to as a total obstruction or a partial obstruction, and a vascular restriction may be referred to as a total restriction or a partial restriction.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

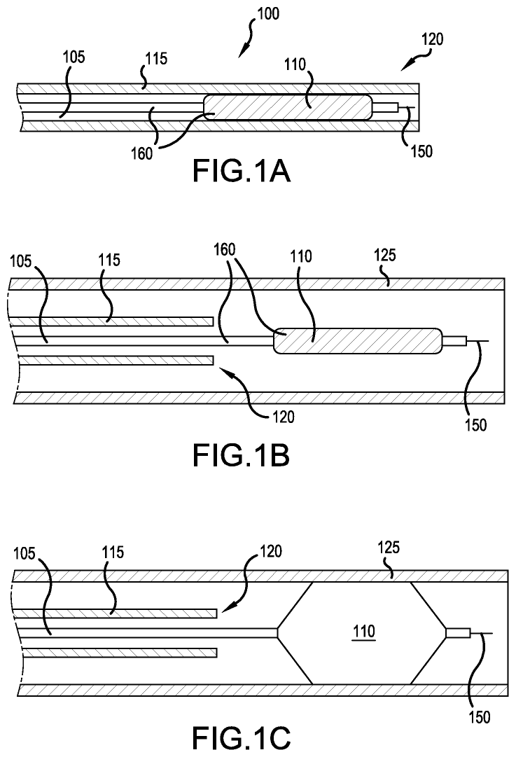

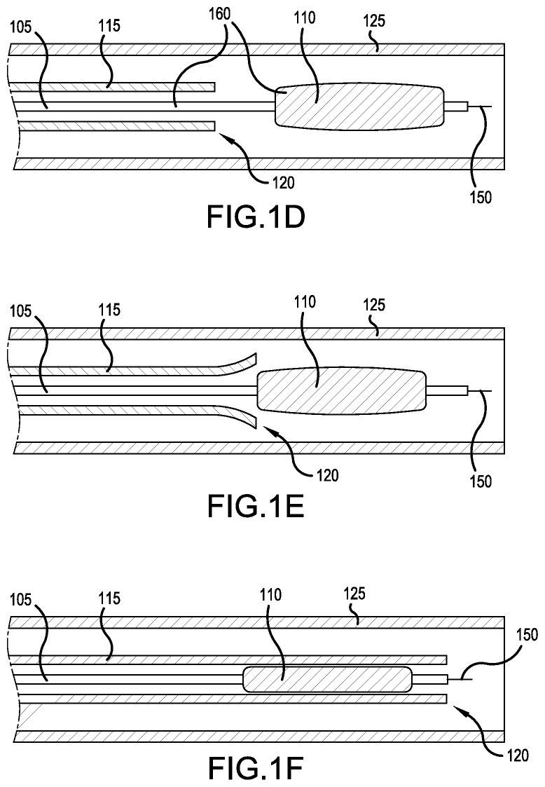

Referring to FIG. 1A, there is depicted an exemplary kit or system 100, wherein the kit or system 100 comprises an outer sheath 115 and a balloon catheter 160 and an optional guidewire 150. The balloon catheter 160 includes a shaft 105 and an expandable member, namely a drug-coated balloon (DCB) 110, coupled to a distal portion of the shaft 105. This FIG. 1A, depicts the shaft 105 and the DCB 110, which is in a collapsed state, within the outer sheath 115. That is, both the shaft 105 and the distal end of DCB 110 are disposed proximally of the distal end 120 of the outer sheath 115 so that DCB 110 can remain covered during introduction and delivery of the DCB 110 to the vascular site, thereby preventing removal or loss of the drug or therapeutic agent, which is on the DCB 110.

During manufacturing or packaging, after the drug or therapeutic is applied to the DCB 110, the DCB 110 is spirally wound and/or folded in a collapsed state and inserted into the outer sheath 115 so that the DCB 110 is completely covered by the outer sheath 115. Because the insertion of the DCB 110 into the outer sheath 115 during manufacturing, which is a controlled environment, the diameter of the collapsed DCB 110 is able to be reduced to a size smaller than the orifice and lumen of the outer sheath 115.

Referring to FIG. 1B, there is depicted the exemplary kit or system 100 within a subject's vasculature 125. Particularly, this figure depicts the collapsed DCB 110 extending beyond the distal end 120 of the outer sheath 115. Referring to FIG. 1C, the DCB 110 is still extending beyond the distal end 120 of the outer sheath 115, but the DCB 110 is in an expanded state adjacent the vasculature 125. As the DCB 110 expands, it compresses a vascular obstruction or restriction (not shown) against the vascular wall(s) 125, thereby widening the opening or passageway within the vasculature 125 at the location where the obstruction or restriction exits.

Referring to FIG. 1D, this figure depicts the collapsed DCB 110 extending beyond the distal end 120 of the outer sheath 115 after the DCB 110 was expanded in FIG. 1C. The diameter of the DCB 110 in the collapsed state in FIG. 1D is illustrated to greater than the diameter of the DCB 110 in the collapsed state in FIG. 1B because the DCB 110 is tightly wound (or folded) and collapsed prior to insertion in the outer sheath 115 during manufacturing and/or packaging. But after expansion of the DCB 110 in the vasculature 125, it is possible that the DCB 110 may not collapse to its originally packaged size, thereby potentially increasing the difficulty of sliding the outer sheath 115 over the DCB 110. That is, the DCB 110 and outer sheath 115 may possibly abut and contact one another as the physician attempts to slide the re-collapsed/re-deflated DCB 110 back into the outer sheath 115 because the deflated diameter of the DCB 110 may be greater than the orifice of the distal end of the outer sheath 115 and the lumen of the outer sheath 115 extending from the orifice for a predetermined distance (A).

To accommodate for the enlarged deflated diameter of the DCB 110 (in comparison to the originally (packaging) deflated diameter of the DCB 110 prior to inflation) it may be desirable to increase the size of the diameter of the orifice of the distal end of the outer sheath 115 and the lumen of the distal portion end of the outer sheath 115. Referring to FIG. 1E, there is shown a proximal portion of the collapsed DCB 110 that is partially axially located and extending into the radially expanded distal end 120 of the outer sheath 115. After the collapsed DCB 110 is completely axially located and extending into the radially expanded distal end 120 of the outer sheath 115, the orifice and lumen are reduced to their substantially initial sizes, as depicted in FIG. IF.

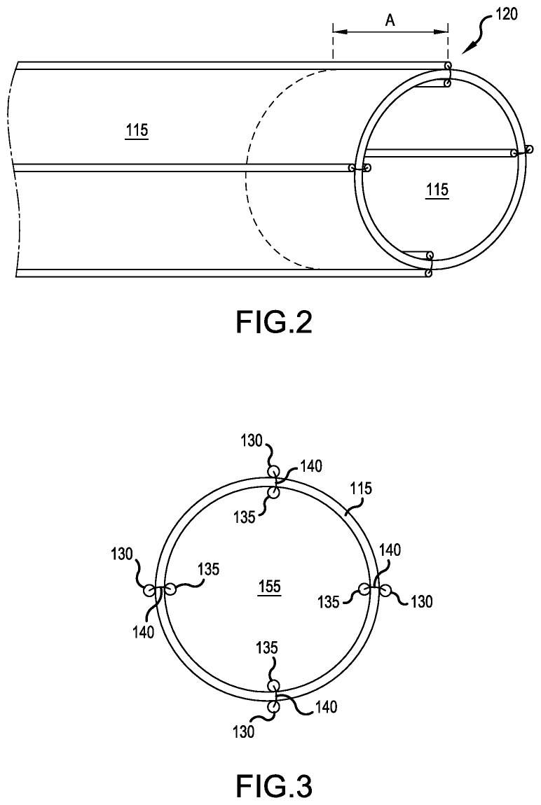

Referring to FIGS. 2 and 3, there is depicted a distal portion of an outer sheath 115 comprising a means for increasing and decreasing the diameter of the orifice 155 and for increasing and decreasing the diameter or size of the lumen, for at least a predetermined length (A) extending proximally from the distal end of the outer sheath 115. In accordance with a representative embodiment, a plurality of wires 140 is radially disposed within the distal portion of the outer sheath 115, and is evenly spaced around a circumference of the distal portion of the outer sheath 115 to increase or decrease the diameter or size of the lumen, for at least a predetermined length (A) extending proximally from the distal end of the outer sheath 115. The outer sheath 115 may also comprise a handle (not shown). The wires 140 are coupled to the handle, and upon activating of the handle (e.g., by pulling or pushing the handle), tension is applied to the wires 140, causing the diameter of the orifice 155 (and the lumen at the distal portion of the outer sheath) to increase to a size equal to or greater than the size of the diameter of the collapsed DCB 110 in FIG. 1D. Also, upon deactivating the handle, tension to the wires 140 is reduced, and the diameter of the orifice 155 decreases to its original size or size substantially similar thereto.

As shown in FIGS. 2 and 3, the wires 140 are wrapped around the distal facing portion of the distal end 120 of the outer sheath 115. One end of each wire 140 is coupled to or extends into the interior of the outer sheath 115 towards a proximal direction, such as towards and including the handle, and the other end of each wire 140 is coupled to or extends from the distal end 120 of the outer sheath 115 towards a proximal direction, such as towards and including the handle. Upon activating (e.g., pulling or pushing the handle), tension is applied to the wires 140, and the diameter of the orifice 155, as well as the lumen at the distal portion of the outer sheath 115 for a predetermined distance (A) increases. FIGS. 2 and 3 depict the wires 140 extending into exterior lumens 130 and interior lumens 135, but one of the wires 140 may be affixed to the distal portion of the sheath with the predetermined distance (A) of the outer sheath 115.

Again, FIGS. 2 and 3 depict the wires 140 extending into the exterior lumens 130 and interior lumens 135, wherein the exterior lumens 130 and the exterior lumens 130 are disposed radially exterior and radially interior of the outer sheath, but the lumens may be integral to the outer sheath 115. Additionally, these figures depict the exterior lumens 130 and interior lumens 135 as evenly radially spaced from the center of the wall of the outer sheath 115, but it may be desirable for the exterior lumen 130 or the interior lumen 135 to be radially offset from the center of the wall of the outer sheath 115, such that the exterior lumen 130 (or the interior lumen 135) is radially further or closer to the center of the wall of the outer sheath 115 relative to the distance that the interior lumen 135 (or the exterior lumen 130) is radially to the center of the outer sheath 115. Furthermore, these figures depict the distal ends of the exterior lumens 130 and interior lumens 135 as both being flush (even) with the distal end of the outer sheath 115, but it may be desirable for either the distal end of the exterior lumen 130 or the distal end of the interior lumen 135 to be axially (longitudinally) offset from the distal end of the wall of the outer sheath 115, such that either the exterior lumen 130 (or the interior lumen 135) is axially further or closer to the distal end of the wall of the outer sheath 115 relative to the distance that the interior lumen 135 (or the exterior lumen 130) is axially to the distal end of the outer sheath 115.

Continuing to refer to FIGS. 2 and 3, the thickness of the outer sheath 115 may reduce from the proximal end of the predetermined distance (A) to the distal end of the predetermined distance (A) such that the wall thickness is the thinnest at the distal end 120 of the outer sheath 115. Alternatively or additionally, the predetermined distance (A) of the outer sheath 115 may be constructed of a more flexible material in comparison to the material with which the remainder of the outer sheath 115 is constructed. Either or both of these design configurations to the predetermined distance (A) may increase the outer sheath's ability to more effectively increase and/or decrease the diameter of the orifice 155 and increase and/or decrease the diameter or size of the lumen, for at least a portion of the predetermined length (A).

Actuating the plurality of wires 140 using a handle is a viable way to cause the diameter of the orifice 155, as well as the lumen at the distal portion of the outer sheath 115 to increase over the predetermined distance (A). Alternatively, and instead of manual actuation of the wires using the referenced handle, the present teachings contemplate effecting the change in diameter over the predetermined distance (A) using a shape-memory metal. In accordance with a representative embodiment, each of the plurality of wires comprises an alloy of Nickel and Titanium having the trade name Nitinol®. As is known, Nitonol° is deformed at a comparatively low temperature, and returns to its original shape when heated through the shape memory effect. Notably, the outer sheath 115 is contemplated to have an inner layer that is includes shape-memory metal that expands, and an outer layer that covers the inner layer. As such, when the outer layer is pulled back, the inner layer expands.

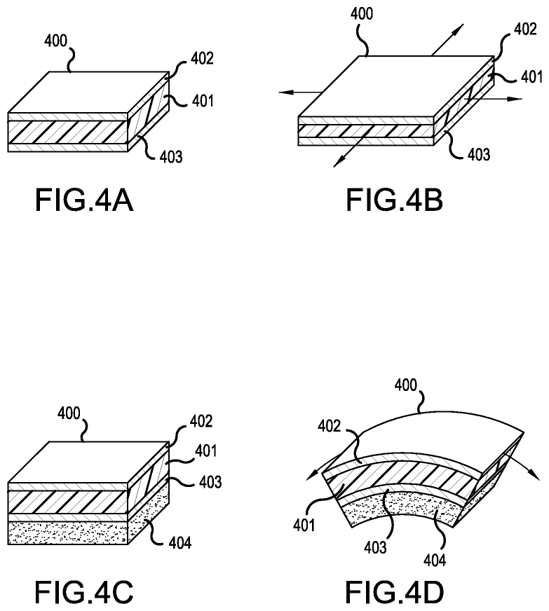

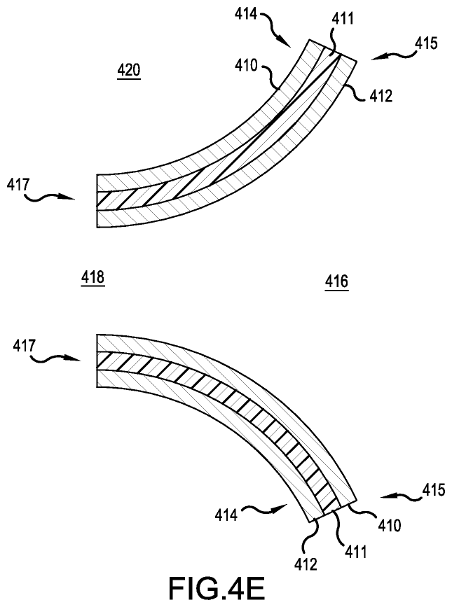

FIGS. 4A-4D depict sections 400 of a structure comprising an ionic electroactive polymer (EAP) contemplated for use in a sheath in accordance with a representative embodiment.

Referring to FIG. 4A, the section 400 comprises a layer of ionic EAP 401 disposed between a first electrode 402 and a second electrode 403. The layer of ionic EAP 401 is a material in which actuation is caused by the displacement of ions inside the polymer. Only a few volts are needed for actuation, but the ionic flow implies a higher electrical power needed for actuation, and energy is needed to keep the actuator at a given position. Examples of ionic EAPs are conductive polymers, ionic polymer-metal composites (IPMCs), and responsive gels. Yet another example is a Bucky gel actuator, which is a polymer-supported layer of polyelectrolyte material consisting of an ionic liquid sandwiched between two electrode layers consisting of a gel of ionic liquid containing single-wall carbon nanotubes. The name comes from the similarity of the gel to the paper that can be made by filtering carbon nanotubes, the so-called buckypaper.

Alternatively, the EAP may be a field driven EAP. Such polymers are described for example in commonly owned U.S. Patent Application Publication No. 20200168783, the disclosure of which is specifically incorporated herein by reference.

Actuation of the layer of ionic EAP 401 is effected by application of a voltage difference between the first and second electrodes, as shown in FIG. 4B. Notably, the arrows depicted in FIG. 4B show the direction of the forces that can be applied upon application of a voltage to the layer of ionic EAP 401 by the first and second electrodes 402, 403. In the present illustration of the properties of the material, when voltages are applied, the layer of ionic EAP 401 its surface area is increased by the migration of ions, representing induced strain to the ionic EAP 401 and the section 400. As such, by application of a voltage to the first and second electrodes, section 400 is stretched, and its constituent layers are thinner than before application of the voltage (i.e., in FIG. 4A).

FIG. 4C depicts the section 400 disposed over a carrier layer 404, the shape of which is desirably changed by application of a voltage to the layer of ionic EAP 401. As described more fully below, in accordance with a representative embodiment, the carrier layer 404 may be part of a catheter used in a balloon angioplasty. However, and as noted above, the present teachings are not limited to applications to balloon catheters, and are more generally applicable to an apparatus comprising a sheath in which an opening or expansion of an orifice is effected.

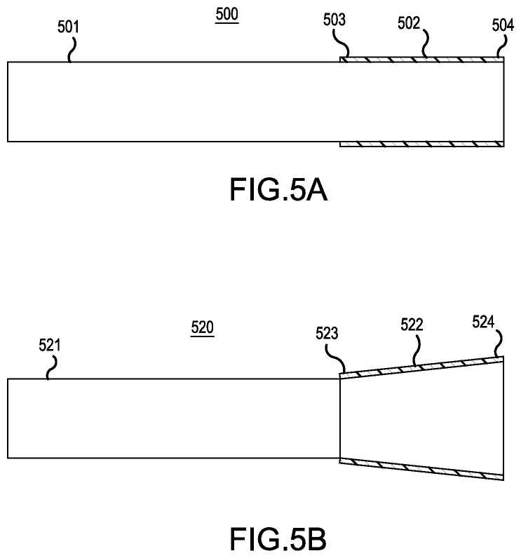

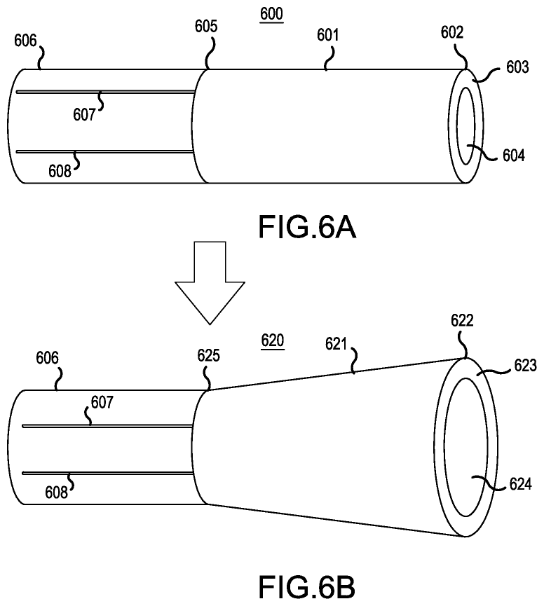

FIG. 4D depicts the section 400 upon application of a voltage between the first and second electrodes 402, 403. As shown, the layer of ionic EAP 401 expands as shown by the arrows, causing the stretching of the first and second electrodes 402, 403, and the carrier layer 404. As such, the carrier layer 404 is deformed in an arcuate manner as shown. As will be appreciated, this bending of the section 400, and thereby the carrier layer 404, is beneficial in the retrieval of balloon catheters such as described above, and below. FIG. 4E shows cross-sectional views of a portion 420 of an apparatus showing prior to and after actuation of an EAP sheath in accordance with a representative embodiment. Specifically, the apparatus comprises a first electrode 410 and a second electrode 412, which sandwich a layer of ionic EAP 411. Again, and only for purposes of illustration, the layer of ionic EAP 411 may be a portion of a balloon catheter (e.g., balloon catheter 160 described above). As will be appreciated from a review of FIG. 4E, a distal portion 414 has a distal end 415 comprising a first orifice 416 having a diameter. Similarly, a proximal end 417 comprises a second orifice 418. As depicted the diameter of the first orifice 416 is greater than the diameter of the second orifice 418. Upon application of a voltage between the first and second electrodes 410, 412, ions within the layer of ionic EAP 411 move to one side. This migration causes that side to swell, and the overall shape of the portion 420 is altered (deformed). By the present teachings, this deformation of structures comprising ionic EAP layers allows for a relative increase in diameter of one (e.g., distal) end relative to another (e.g., proximal) end, which is advantageous in certain applications, such as the retrieving a balloon catheter. In applications to balloon catheter retrieval the actuation of the layer of ionic EAP 411, causes a deformation (bending) of the structure, ultimately increasing the diameter of the distal end relative to the proximal end as shown in FIGS. 5A-6B below.

FIGS. 5A-5B are cross-sectional views of an apparatus 500 comprising of a sheath comprising EAP prior to and after expansion of a distal portion of the sheath in accordance with a representative embodiment. Various aspects details of the representative embodiments of FIGS. 5A-5B are common to the representative embodiments described above in connection with FIGS. 1A-4E. Such common aspects and details may not be repeated in order to avoid obscuring the presently described representative embodiments.

Referring to FIG. 5A, an apparatus 500 comprises a structure 501 having an unactuated sheath 502 disposed along a portion of its length. The structure 501 is illustratively cylindrical in shape, with a hollow middle portion. The unactuated sheath 502 comprises EAP (ionic or field driven) and has a proximal portion 503 and a distal portion 504, and, like the structure 501 is illustratively a hollow cylinder.

Referring to FIG. 5B, an apparatus 520 comprises a structure 521 having an actuated sheath 522 (i.e., unactuated sheath 502 after being actuated by application of a voltage or electric field depending on the type of EAP used) disposed along a portion of its length. The structure 521 is illustratively cylindrical in shape, with a hollow middle portion. The actuated sheath 522 comprises EAP (ionic or field driven) and has a proximal portion 523 and a distal portion 524. As seen in FIG. 5B, through the deformation (in this case bending) action cause by the EAP, and as a result, the actuated sheath 522 expands the structure 521 at its distal portion 524, but not at its proximal portion 523. As such, the diameter of the opening of the distal portion 524 of the structure 521 is greater than its diameter at the proximal portion 523.

FIGS. 6A-6B are perspective views of an apparatus 600, 629 comprising a sheath comprising EAP prior to and after expansion of a distal portion of the sheath in accordance with a representative embodiment. Various aspects details of the representative embodiments of FIGS. 6A-6B are common to the representative embodiments described above in connection with FIGS. 1A-5B. Such common aspects and details may not be repeated in order to avoid obscuring the presently described representative embodiments.

Referring to FIG. 6A an unactuated sheath 601 is disposed over a shaft 606, which may be hollow. The unactuated sheath 601 comprises a distal portion 602 having a distal end 603, and a proximal portion 605. A first orifice 604 having a first diameter is disposed at the distal end 603.

A first electrode 607 and a second electrode 608 are disposed along an outer portion of the shaft 606, and extend along its length to make contact with the unactuated sheath 601.

Referring to FIG. 6B an actuated sheath 621 (i.e., unactuated sheath 601 actuated by application of a voltage to the first and second electrodes 607, 608) is disposed over the shaft 606. The actuated sheath 621 comprises a distal portion 622 having a distal end 623, and a proximal portion 625. A second orifice 624 having a second diameter is disposed at the distal end 623. As shown, upon application of the voltage to first and second electrodes 607, 608, the distal portion 622 expands relative to the proximal portion 625. As a result through the deformation (in this case bending) action cause by the EAP, the actuated sheath 621 causes a second orifice 624 to be formed at the distal end 623. As shown, the second diameter of the second orifice 624 is greater than the first diameter of the first orifice 604 having the unactuated sheath 601 disposed around the shaft 606.

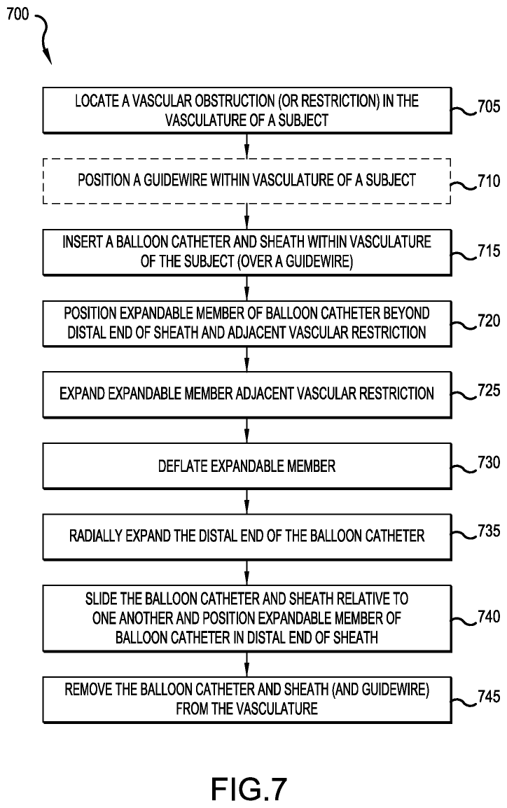

FIG. 7 is a representative flow diagram of a method of performing an angioplasty procedure on a subject using the kit or system illustrated in FIG. 1A.

Referring to FIG. 7, there is depicted a flow chart illustrating the steps of a method 700 of using, for example, the kit or system 100 (depicted in FIG. 1A) for treating a vascular obstruction or restriction by performing an angioplasty procedure in a human subject's vascular system. Method 700 in FIG. 7 includes locating the vascular obstruction or restriction in the vasculature of a subject at step 705. The next step 710 is optional and includes the step of positioning a guidewire within the vasculature. The guidewire may be used to guide the introduction and direct the placement of the balloon catheter and outer sheath within the vasculature. Assuming a guidewire is used, the next step 715 includes positioning the outer sheath and the balloon catheter within the vascular system of the subject approximate the restriction, wherein the balloon catheter comprises a shaft and an expandable member, wherein the outer sheath comprises a distal portion and a distal end and an orifice at the distal end and a lumen extending proximally from the orifice, wherein the orifice has a first diameter.

After positioning the outer sheath and the balloon catheter approximate the restriction, the expandable member is extended distally of the distal end of the outer sheath and is positioned adjacent the restriction in step 720. Next, the expandable member is expanded adjacent the restriction in step 725. It is preferable that the expandable member is a DCB. So, after the DCB is inflated for a predetermined period of time to reduce the size of the restriction and introduce the therapeutic agent to the remainder of the restriction, the expandable member is deflated in step 730.

Next, in step 735, the orifice at the distal end of the outer sheath is increased in size to a second diameter, wherein the second diameter is greater than the first diameter, in order to accommodate the increased size of the deflated inflatable member after the inflatable member has been inflated and deflated. While the size of the orifice and the distal portion of the outer sheath are increased to a diameter equal to or greater than the size of the deflated inflatable member, the expandable member is inserted through the orifice and into the distal end of the sheath in step 740. After the expandable member is in the distal end of the outer sheath, the orifice at the distal end of the outer sheath is radially reduced in size and both the outer sheath and balloon catheter are simultaneously removed from the vasculature in step 745.

The present disclosure, in various aspects, embodiments, and configurations, includes components, methods, processes, systems and/or apparatus substantially as depicted and described herein, including various aspects, embodiments, configurations, sub combinations, and subsets thereof Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, for example, for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating a restriction within a subject's vascular system, the method comprising:
locating a restriction in the vascular system of the subject;
positioning a sheath and a balloon catheter within the vascular system of the subject approximate the restriction, wherein the balloon catheter comprises a shaft and an expandable member, wherein the sheath comprises a distal portion and a distal end and an orifice at the distal end and a lumen extending proximally from the orifice, wherein the orifice has a first diameter;
positioning the expandable member distally of the distal end of the sheath and adjacent the restriction within the vascular system;
expanding the expandable member adjacent the restriction;
deflating the expandable member;
radially enlarging the orifice at the distal end of the sheath to a second diameter, wherein the second diameter is greater than the first diameter;
inserting the expandable member into the distal end of the sheath while the orifice has the second diameter; and
radially reducing the orifice at the distal end of the sheath.

2. The method of claim 1, wherein the expandable member comprises a length, wherein the distal portion of the sheath comprises a predetermined length, whereupon inserting the expandable member into the distal end of the sheath, the length of expandable member is disposed within the lumen of the sheath.

3. The method of claim 2, wherein the distal portion of the sheath comprises a predetermined length, whereupon inserting the expandable member into the distal end of the sheath, the length of expandable member is disposed within the predetermined length of the distal portion of the sheath.

4. The method of claim 3, wherein the predetermined length of the sheath is greater than the length of expandable member.

5. The method of claim 4, wherein radially reducing the orifice at the distal end of the sheath comprises radially reducing the orifice at the distal end of the sheath to a third diameter.

6. The method of claim 5, wherein the third diameter is less than the second diameter.

7. The method of claim 6, wherein the third diameter is substantially the same as the first diameter.

8. The method of claim 1 further comprising the step of simultaneously removing the sheath and balloon catheter from the vascular system of the subject.

9. A catheter system comprising:
a balloon catheter comprising an expandable member;
a sheath comprising a distal portion having a distal end, wherein the distal end comprise an orifice having a diameter, wherein the distal portion comprises a lumen extending proximally from the orifice, and
a means for increasing and decreasing the diameter of the orifice.

10. The catheter system of claim 9, wherein the means for increasing and decreasing the diameter of the orifice comprises a plurality of wires radially disposed within the distal portion of the outer sheath and evenly spaced around a circumference of the distal portion of the outer sheath and extending proximally.

11. The catheter system of claim 10, wherein the outer sheath comprises a handle, wherein the wires are coupled to the handle, and whereupon activating the handle, tension is applied to the wires, and the diameter of the orifice increases.

12. The catheter system of claim 11, whereupon deactivating the handle, tension is reduced to the wires, and the diameter of the orifice decreases.

13. The catheter system of claim 10, wherein the plurality of wires each comprises a shape-memory metal configured to cause the increasing and decreasing of the diameter of the orifice.

14. An apparatus, comprising:
a sheath having a distal portion comprising a distal end, the distal end comprising a first orifice having a first diameter, wherein the sheath comprises an electroactive polymer (EAP), which when actuated, increases the first diameter of the first orifice.

15. The apparatus of claim 14, wherein the sheath has a proximal portion comprising a proximal end, the proximal end comprising a second orifice having a second diameter, wherein the EAP, when actuated, causes no increase or decrease in the second diameter.

16. The apparatus of claim 15, wherein, when actuated, the sheath has a flared profile between the first orifice and the second orifice.

17. The apparatus of claim 14, wherein the EAP is an ionic EAP.

18. The apparatus of claim 14, wherein the EAP is a field-driven EAP.

19. The apparatus of claim 14, further comprising a carrier layer adapted to expand upon actuation of the sheath.

20. The apparatus of claim 14, wherein the carrier layer is a catheter.
